(19) United States
(12) Patent Application Publication
(10) Pub. No.: US 2020/0131579 A1
(43) Pub. Date: Apr. 30, 2020

(54) IMPROVED METHOD FOR PROVIDING INFORMATION ON BREAST CANCER AND DIAGNOSTIC KIT THEREFOR

(71) Applicants: OPTIPHARM.CO.,LTD., Cheongju-si (KR); Yonsei University Wonju Industry-Academic Cooperation Foundation, Gangwon-do (KR)

(72) Inventors: Hye Young LEE, Wonju-si (KR); Hye Young WANG, Gunsan-si (KR); Yeun KIM, Wonju-si (KR)

(21) Appl. No.: 15/121,377

(22) PCT Filed: Feb. 28, 2014
(86) PCT No.: PCT/KR2014/001697

§ 371 Date: Aug. 24, 2016

(30) Foreign Application Priority Data
Feb. 25, 2014 (KR) 10-2014-0022078

(51) Int. Cl. C12Q 1/6886; C12Q 1/6806; C12N 15/10

(52) U.S. Cl. CPC C12Q 1/6886; C12Q 1/6806; C12Q 2600/158; C12Q 2600/16; C12N 15/1096

(57) ABSTRACT

The present disclosure relates to an improved method for providing information on breast cancer and a diagnostic kit therefore, and more specifically, to an improved kit for diagnosing breast cancer by using a one-tube nested PCR and a diagnostic method therefor.

### CROSS-REFERENCE TO RELATED APPLICATIONS

This application is based on and claims priority from Korean Patent Application No. 1020140022078, filed on Feb. 25, 2014, with the Korean Intellectual Property Office, the disclosure of which is incorporated herein in its entirety by reference.

### TECHNICAL FIELD

The present disclosure relates to an improved method for providing information on breast cancer and a diagnostic kit therefor and, more specifically, to an improved kit for diagnosing breast cancer by using a one-tube nested PCR and a diagnosing method therefor.

### BACKGROUND

Breast cancer is caused through multiple genetic variations in each individual cell, and metastasis is active multiple processes including forming angiogenesis and the like and includes processes of an interstitial tissue around cancer cells, lymphatic, local invasion to blood vessels, penetration to a blood flow, and movement to other organs, and the like. Even though the size of tumor is small, the spread of cancer cells can be detected, and even after removing primary tumor by operating, the cancer cells may remain in lymphatic system or a blood flow, and thus these cancer cells which cannot be found by a typical method are considered as the cause of the recurrence of the breast cancer. To this reason, finding the breast cancer cells in the peripheral blood of the breast cancer patient is magnified as an important prognostic factor for predicting the survival of the patient, and thus, considered to become a criterion for selecting secondary treatment methods and a useful method of follow-up survey after primary treatment. However, a direct cell test for a blood sample has very low diagnostic sensitivity, and the immunochemical test is performed, but also has low diagnostic sensitivity, and in some cases, false-positive results are obtained.

A human epidermal growth factor receptor 2 (HER2) is a glycoprotein of 185 kDa having a tyrosine kinase activity and plays an important role to activate a signaling system under cells which regulate growth and differentiation of epithelial cells. In breast cancer patients, amplification of the HER2 gene and overexpression of the HER2 protein are observed in 10 to 34% of the breast cancer patients. The analysis for the HER2 state is important in prognosis of the patients and treatment of trastuzumab (Herceptin, Roche) which is an anti-HER2 monoclonal antibody. In early days, as a method of determine whether the amplification of the HER2 gene or overexpression of the protein, Southern or Western blotting is used, but is not clinically applied thereto. A immunohistochemical staining method (IHC) is most widely used as a primary screening test, there is a difference for each organ, and technical accuracy or reproduction of the result is controversial (Press M F, Sauter G, Bernstein L, Villalobos I E, Mirlacher M, Zhou J Y, et al. Clin Cancer Res 2005; 11:6598-607.). A fluorescence in situ hybridization (FISH) method is known to be the most reliable, and has advantages of being performed in a paraffin embedded tissue because DNA itself is very stable, not being sensitive to a state of the tissue compared with the IHC, and having high read concordance between pathologists. However, it is known that the method has an inconvenience that the testing process is complicated and needs to be performed through a fluorescence microscope in the dark when reading, has a disadvantage that permanent preservation of the result is impossible because the fluorescence is used, and further, cannot be performed in small-scale hospitals and the like because the value of the fluorescent probe is very expensive (Lewis F, Jackson P, Lane S, Coast G, Hanby A M. Histopathology 2004; 45: 207-17.). The HER2 gene amplification starts to be known to be associated with invasion of tumor and bad prognosis Re @ villion F, Bonneterre J, Peyrat J P. Eur J Cancer 1998; 34:791-808.), but starts to clinically significantly receive attention since relevance with anti-cancer treatment and trastuzumab treatment is found. A patient group having HER2 gene amplification is subject to a molecular targeted therapy targeting HER2. Currently, the most accurate HER2 status criteria is considered as the FISH, but since there is a limit that a lot of costs and time are required and institutions having test capacity are insufficient, the method is not widely diffused.

Researches which find micrometastasis of breast cancer cells in peripheral blood or bone marrow of the breast cancer patient gradually receive attention, and verifying the micrometastasis of the breast cancer cells before and after surgery regardless of the stage of the patient in the clinical management of the patient will be an important factor in terms of diagnosis or tracking management after surgery. Recently, a reverse transcription-polymerase chain reaction (RT-PCR) method for a cancer cell-specific mRNA has been researched as a method having very high diagnostic sensitivity while being very useful to diagnose fine residual cancer in the peripheral blood or the bone marrow of many kinds of cancer patients (Ghossein R A, Juan R. Cancer 1996; 78:10-6.).

### SUMMARY

The present disclosure has been made in an effort to provide an improved method for providing information for diagnosis of breast cancer

Further, the present disclosure has been made in an effort to provide a kit for diagnosing breast cancer.

An exemplary embodiment of the present disclosure provides a method for providing information for diagnosis of breast cancer, in which the method includes: a) isolating a total RNA from cells obtained from the blood of a cancer suspected patient; b) synthesizing cDNA from the isolated total RNA; c) performing a real-time PCR of the synthesized cDNA by using one or more primer sets and probes selected from the group consisting of a primer set and a probe capable of amplifying a human epidermal growth factor receptor 2 HER2 and a primer set and a probe capable of amplifying glyceraldehyde-3-phosphate dehydrogenase (GAPDH); and d) comparing the amplified level with an expressed level in a normal person, in which the primer set capable of amplifying the HER2 is a primer set selected from the group consisting of primer sets as set forth in SEQ ID NOS. 1 and 2, SEQ ID NOS. 3 and 4, SEQ ID NOS. 6 and 7. and SEQ ID NOS. 8 and 9 or a mixture of these primer sets and the probe is one or more of probes as set forth in SEQ ID NO. 5, SEQ ID NOS. 10 and 11.

In an exemplary embodiment of the present disclosure, the comparing of the amplified level with the amplified level in the normal person may be preferably performed by a standard or cut-off value, but the present disclosure is not limited thereto.

In another exemplary embodiment of the present disclosure, the primer set capable of amplifying the GAPDH is set forth in SEQ ID NOS. 12 and 13 and the probe has a base sequence as set forth in SEQ ID NO. 14, preferably, but the present disclosure is not limited thereto.

Another exemplary embodiment of the present disclosure provides a composition for diagnosing breast cancer, in which the composition includes: a primer sets capable of amplifying a HER2 which is selected from the group consisting of primer sets as set forth in SEQ ID NOS. 1 and 2, SEQ ID NOS. 3 and 4, SEQ ID NOS. 6 and 7, and SEQ ID NOS. 8 and 9 or a mixture of these primer sets; and a primer set and a probe capable of amplifying GAPDH as active ingredients.

in an exemplary embodiment of the present disclosure, a 5′-terminal of the probe may be marked with a fluorescent material, but the present disclosure is not limited thereto.

Yet another exemplary embodiment of the present disclosure provides a kit for diagnosing breast cancer including the composition of the present disclosure.

According to the exemplary embodiments of the present disclosure, it is possible to detect a slight amount compared with a method of detecting proteins because of using a gene amplification method using HER 2 mRNA based on a real-time RT-PCR method capable of deriving easily quantitative result and provide an inexpensive test method because of not using an antigen-antibody reaction. Further, it is possible to verify that the sensitivity is higher than that of existing known sequences and more easily verify a result because there is no step of verifying bands using electrophoresis. Further, it is possible to enhance the sensitivity in the one tube nested RT-qPCR method itself of the present disclosure as compared with the result of the single RT-qPCR or the multiplex RT-qPCR.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In FIGS. 1 to 16, the present disclosure was performed by using primer & probe base sequences prepared by targeting two sites of the front part (sites 605 to 612; FIGS. 1 to 7) and the middle part (site 2.7 kb-4; FIGS. 8 to 16) in HER2 genes (4.6 Kb) as a base sequence site used in the present disclosure.

FIGS. 17 and 18 illustrate a comparison of HER2 expression levels using a cell line with base sequences at different sites of the front part.

FIGS. 19 and 20 illustrate a comparison of HER2 expression levels using a cell line.

FIGS. 21 and 22 illustrate a comparison of HER2 expression levels using a primer and a probe P1 at site 2725.

FIGS. 23 and 24 illustrate a comparison of HER2 expression levels using a primer and a probe P2 at site 2725.

FIGS. 25 and 26 illustrate a comparison of HER2 expression levels using a primer and a probe P1-2mix at site 2725.

FIGS. 27 and 28 illustrate a comparison of HER2 expression levels using a primer and a probe P1 at site 2740.

FIGS. 29 and 30 illustrate a comparison of HER2 expression levels using a primer and a probe P2 at site 2740.

FIGS. 31 and 32 illustrate a comparison of HER2 expression levels using a primer and a probe P1+2mix at site 2740.

FIGS. 33 and 34 illustrate a comparison of HER2 expression levels using base sequences at the front part (605) and the middle part (site 2725).

FIGS. 35 and 36 illustrate a comparison of HER2 expression levels using base sequences at the front part (612) and the middle part (site 2725).

FIGS. 37 and 38 illustrate a comparison of HER2 expression levels using base sequences at the front part (605) and the middle part (site 2740).

FIGS. 39 and 40 illustrate a comparison of HER2 expression levels using base sequences at the front part (612) and the middle part (site 2740).

FIGS. 41 and 42 illustrate a comparison of HER2 expression levels of one-tube nested RT-qPCR using a base sequence at the front part (605 to 612).

FIGS. 43 to 45 illustrate a comparison of HER2 expression levels of one-tube nested RT-qPCR using a base sequence at the middle part (2725 to 2740).

FIGS. 46 to 48 illustrate a comparison of HER2 expression levels of one-tube nested RT-qPCR using a base sequence obtained by mixing the front part (605 to 612) and the middle part (2725 to 2740).

FIGS. 49 and 50 illustrate a comparison of HER2 expression levels of one-tube nested RT-qPCR using a base sequence obtained by mixing the front part (605 to 612) and the middle part (2725).

FIGS. 51 and 52 illustrate a comparison of HER2 expression levels of one-tube nested RT-qPCR using a base sequence obtained by mixing the front part (605 to 612) and the middle part (2740).

FIG. 53 illustrates a comparison of HER2 mRNA expression levels using breast cancer cell lines.

FIGS. 54 to 56 illustrate a comparison of clinical expression levels of single RT-qPCR according to an IHC-FISH result.

FIGS. 57 to 59 illustrate an ROC curve analysis method for clinical Cut-off determination.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

The method of isolating the total RNA and the method of synthesizing cDNA from the isolated total RNA which are generally used may be performed through known methods, and the detailed description for the process is described in Joseph Sambrook et al., Molecular Cloning, A Laboratory Manual, Cold Spring Harbor Laboratory Press, Cold Spring Harbor, N.Y. (2001); Noonan, K. F., and the like and may be inserted as the reference of the present disclosure.

The primer of the present disclosure may be chemically synthesized by using a phosphoramidite solid support method or other well-known methods. The nucleic acid sequence may be modified by using many means known in the art. As an unlimited example of the modification, there are methylation, “capsulation”, substitution to analogues of one or more natural nucleotides, and modification between nucleotides, for example, modification to a non-charged connection body (For example, methyl phosphonate, phosphotriester, phosphoramidate, carbamates, and the like) or a charged connection body (for example, phosphorothioate, phosphorodithioate, and the like). The nucleic acid may include one or more additional covalently-linked residues, for example, a protein (for example, nucleases, toxins, antibodies, signal peptides, poly-L-lysine, and the like), an intercalating agent (for example, acridine, psoralen, and the like), a chelating agent (for example, metal, radioactive metal, iron, oxidative metal, and the like), and an alkylating agent. The nucleic acid sequence of the present disclosure may be modified by using a marker which may directly or indirectly provide a detectable signal. An example of the marker includes a radioisotope, a fluorescent molecule, biotin, or the like.

In the method of the present disclosure, the amplified target sequence (HER 2 and GAPDH genes) may be marked by a detectable marking material. In an exemplary embodiment, the marking material may be a fluorescent, phosphorescent, chemiluminescent, or radioactive material, but the present disclosure is not limited thereto. Preferably, the marking material may be fluorescein, phycoerythrin, rhodamine, lissamine, Cy-5 or Cy-3. When real-time RT-PCR is performed by marking Cy-5 or Cy-3 in a 5′-terminal and/or a 3′-terminal of the primer when amplifying the target sequence, the target sequence may be marked with the detectable fluorescent marking material.

Further, in the marker using the radioactive material, when the radioisotope, such as ³²P or ³⁵S during the real-time RT-PCR is added in a PCR reaction solution, the amplified production is synthesized and the radioactive material is inserted to the amplified product, and thus, the amplified product may be marked with the radioactive material. One or more oligonucleotide primer sets used for amplifying the target sequence may be used.

The marking may be performed by various methods which are generally performed in the art, for example, a nick translation method, a random priming method (Multiprime DNA labelling systems booklet, “Amersham” (1989)), and a keynation method (Maxam & Gilbert, Methods in Enzymology, 65:499 (1986)). The marking provides a signal which is detectable by fluorescence, radioactivity, color measurement, weight measurement, X-ray diffraction or absorption, magnetism, enzymatic activity, mass analysis, binding affinity, hybridization high frequency, and nanocrystalline.

According to an aspect of the present disclosure, in the present disclosure, an expression level at an mRNA level is measured through RT-PCR. To this end, a new primer set which is specifically bound to the HER 2 and GAPDH genes and a probe marked with fluorescence are required, and in the present disclosure, the corresponding primer and probe specified with a specific base sequence may be used, but the present disclosure is not limited thereto. The primer and probe may be used without limitation so long as performing the real-time RT-PCR by providing a detectable signal which is specifically bound to the genes. Herein, FAM anal Quen (Quencher) mean fluorescent dyes.

The real-time RT-PCR method applied to the present disclosure may be performed through a known process which is generally used in the art.

The process of measuring the mRNA expression level may be used with limitation as long as measuring the mRNA expression level generally and may be performed through radiation measurement, fluorescence measurement or phosphorescence measurement according to a kind of used probe marker, but the present disclosure is not limited thereto.

As one of the method of detecting the amplified product, in the phosphorescence measurement method, when the real-time RT-PCR is performed by marking Cy-5 or Cy-3 in the 5′-terminal of the primer, the target sequence is marked with a detectable fluorescent marker, and the marked fluorescence may be measured by using a fluorescence meter. Further, in the radioactive measurement method, when the real-time RT-PCR is performed, after the amplified product is marked by adding the radioisotope such as ³²P or ³⁵S in a PCR reaction solution, the radioactive material may be measured by using radiation measuring equipment, for example, a Geiger counter or a liquid scintillation counter.

According to an exemplary embodiment of the present disclosure, the probe marked with the fluorescence is attached to the PCR product amplified through the real-time RT-PCR to emit fluorescence having a specific wavelength. Simultaneously with amplification, in the fluorescence meter of the real-rime PCR device, the mRNA expression level of the genes of the present disclosure is measured in real time, the measured value is calculated and visualized through PC and thus, a checker may easily check the expression level.

According to another aspect of the present disclosure, the diagnosis kit may be a kit for a breast cancer diagnosis comprising a required element required for performing a reverse transcription polymerase reaction. The reverse transcription polymerase reaction kit may include each gene-specific primer set of the present disclosure. The primer is a nucleotide having a specific sequence to a nucleic acid sequence of each marker gene and may have a length of approximately 7 by to 50 bp, and more preferably a length of approximately 10 bp to 30 bp.

Other reverse transcription polymerase reaction kits may include a test tube or another suitable container, a reaction buffer (pH and magnesium concentration are varied), deoxynucleotide (dNTPs), enzymes such as Taq-polymerase and reverse transcriptase, DNAse, RNAse inhibitor, DEPC-water, sterile water, and the like.

In the present disclosure, the term “information providing method for cancer diagnosis” is to provide objective basic information required for the diagnosis of cancer as a preliminary step and clinical determination or opinions of doctors are excluded.

The term “primer” means a short nucleic acid sequence which may form a complementary template and a base pair as a nucleic acid sequence having a short free 3-terminal hydroxyl group and serves as a starting point for duplicating the template strand. The primer may initiate DNA synthesis under a presence of a reagent for polymerization (that is, DNA polymerase or reverse transcriptase) and four different nucleoside triphosphates in a suitable buffer solution and at a suitable temperature. The primer of the present disclosure is sense and antisense nucleic acid having 7 to 50 nucleotide sequences as each marker gene-specific primer. The primer may combine an additional feature without changing a basic property of the primer acting as an initial point of the DNA synthesis.

The term “probe” is a single chain nucleic acid molecule and includes a complementary sequence to a target nucleic acid sequence.

The term ‘real-tune RT-PCR’ is a molecular biological polymerization method of reverse-transcribing RNA to complementary DNA (cDNA) by using a reverse transcriptase, amplifying a target by using a target probe including a target primer and a marker by using the made cDNA as a template, and simultaneously, quantitatively detecting a signal generated in the marker of the target probe in the amplified target.

Hereinafter, the present disclosure will be described.

In the present disclosure, the HER2 expression rate in the breast cancer patient was verified by using mRNA RT-qPCR and further, the present disclosure was performed by comparing sensitivity with single PCR and comparing a HER2 expression rate in the breast cancer patient by using a one-tube nested RT-qPCR method in order to enhance the sensitivity to help in more effective treatment and diagnosis of the breast cancer through HER2 expressed in a tissue object and the blood and an expression aspect of the cancer-related marker in the blood for effective treatment.

Hereinafter, the present disclosure will be described.

Base sequences used in the present disclosure are illustrated in FIG. 1.

Comparison of HER2 Expression Levels for Each Cell Line Using RT-qPCR

(1) Comparison of HER2 Expression Levels for Each Cell Line Using Single RT-qPCR

i) Comparison of HER2 expression levels for each cell line using base sequence (sites 605 to 612) of the front part

The expression sensitivity of HER2 was verified by using SK-BR-3 and MCF-7 which were HER2 positive breast cancer cell lines.

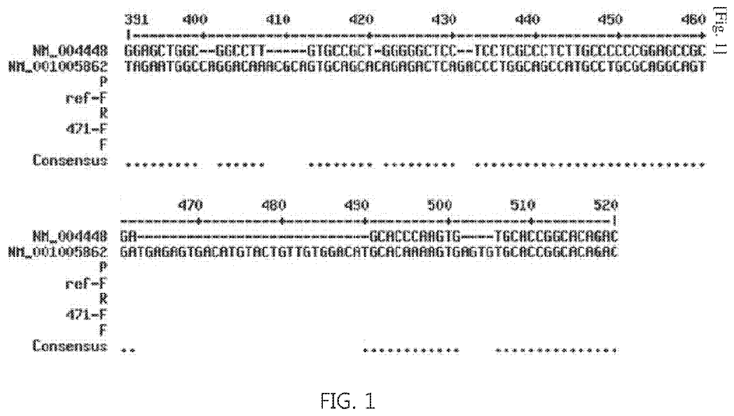

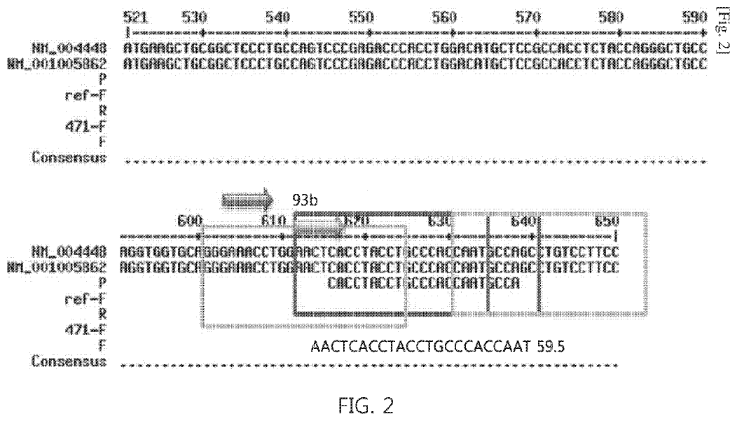

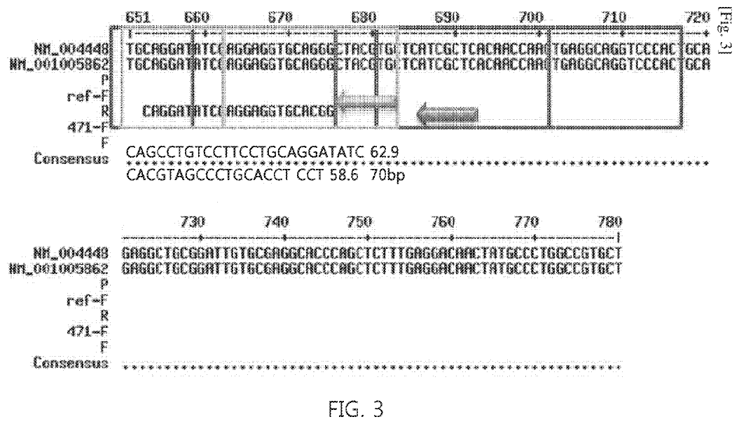

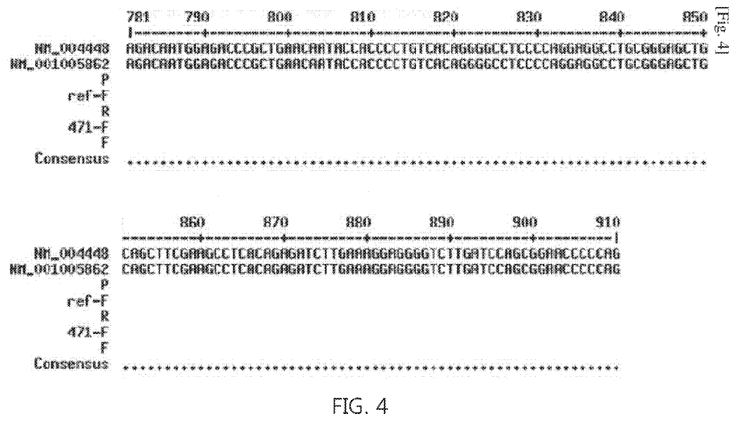

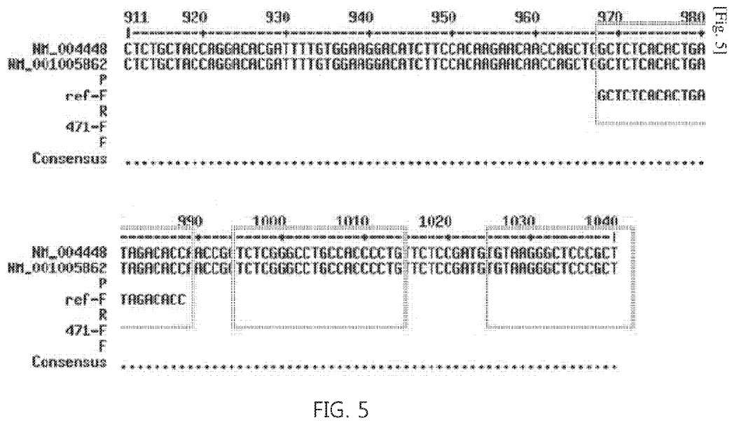

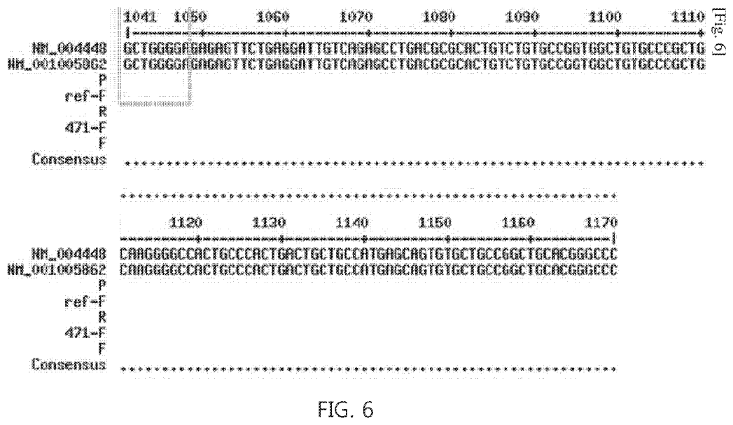

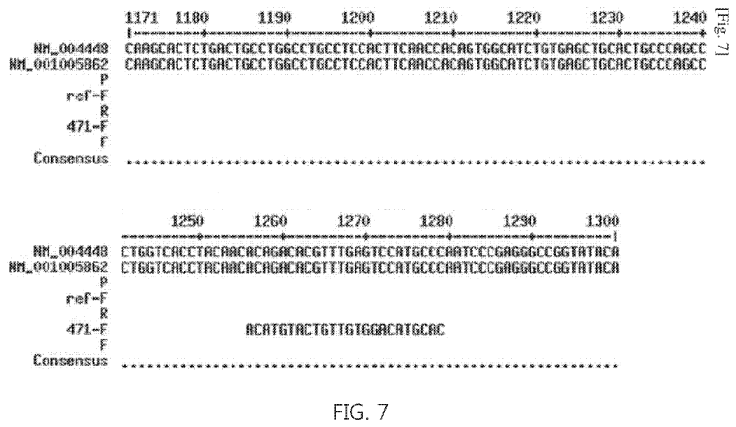

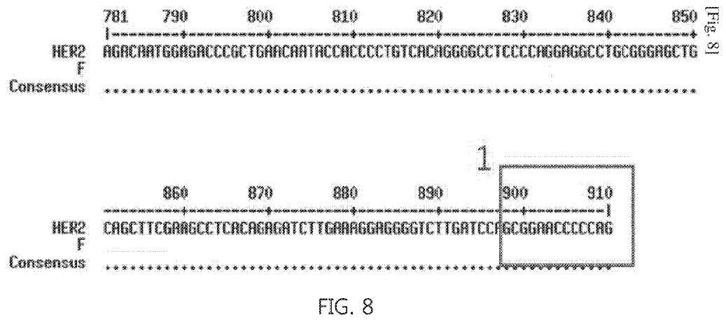

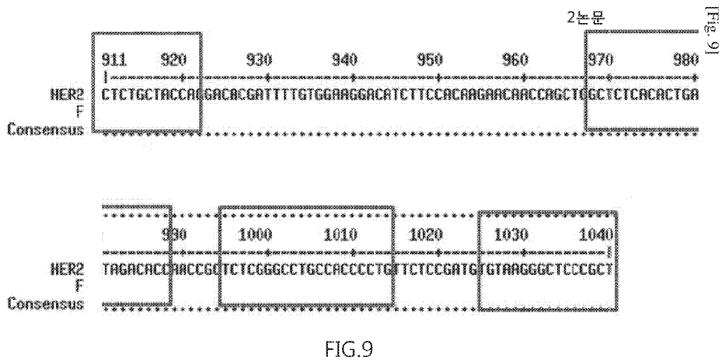

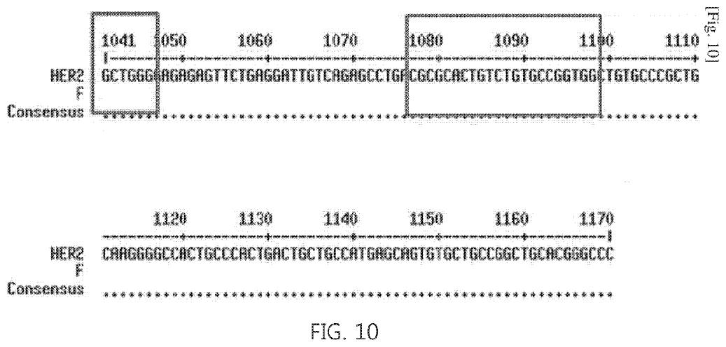

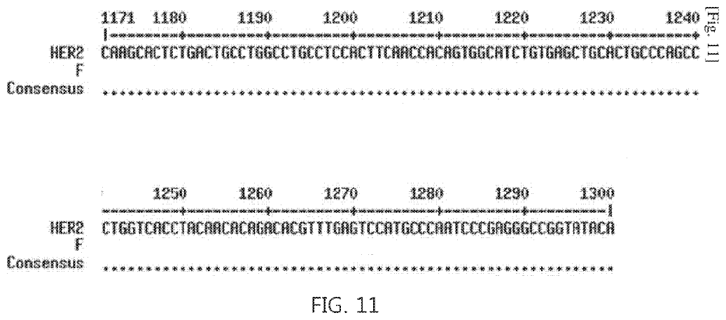

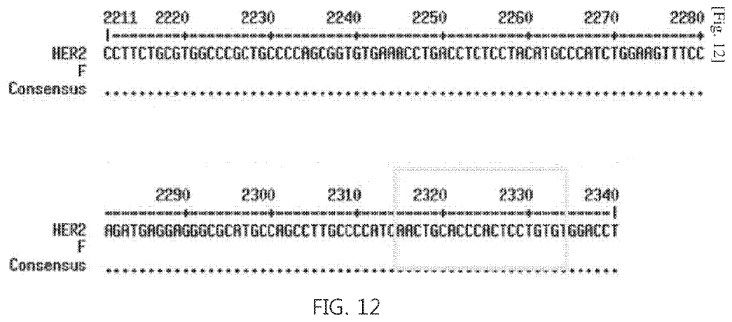

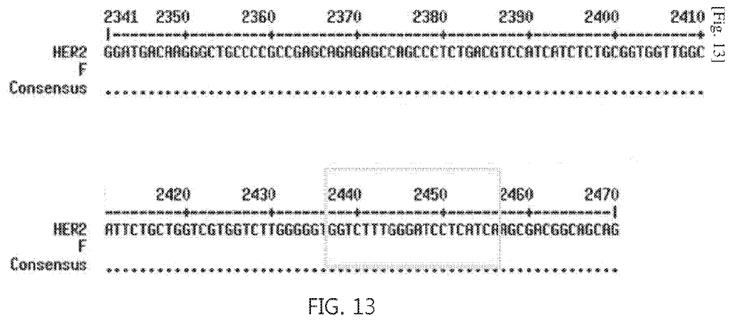

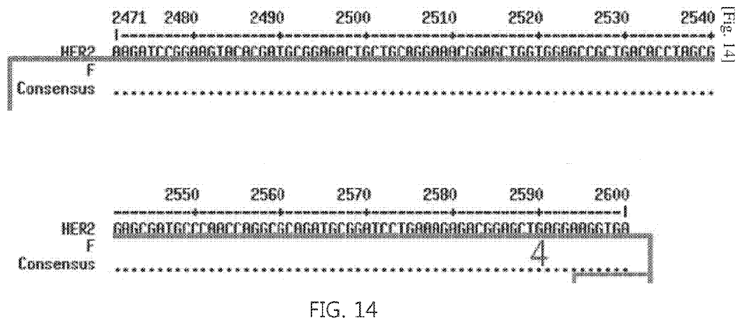

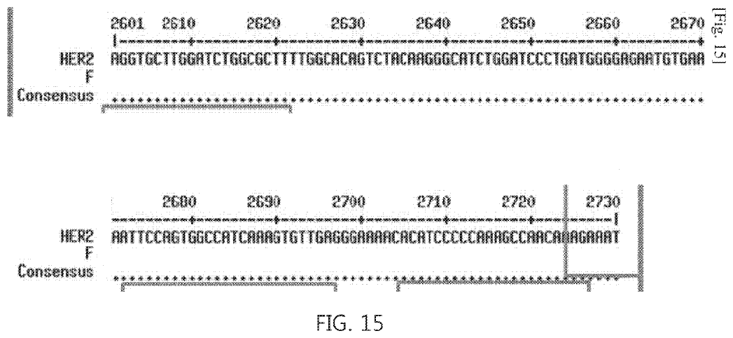

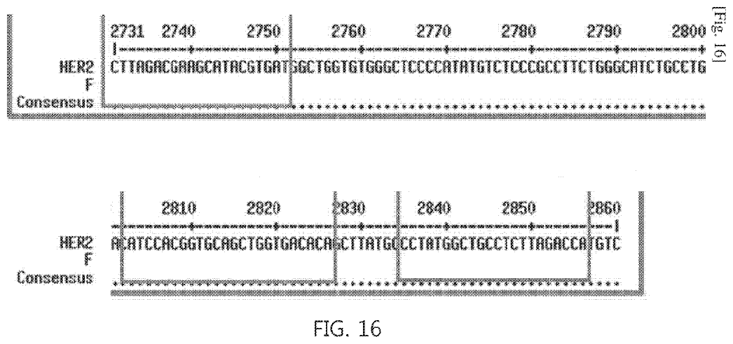

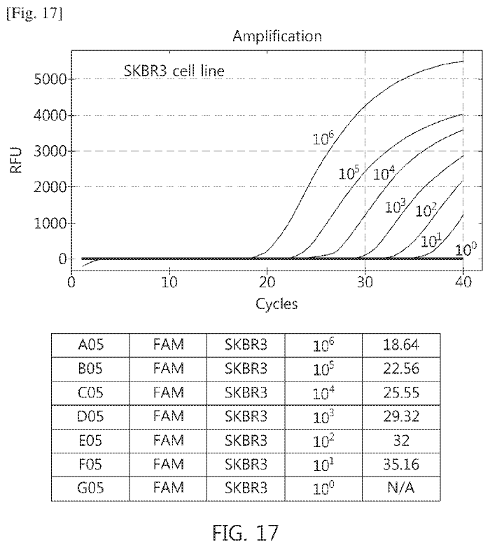

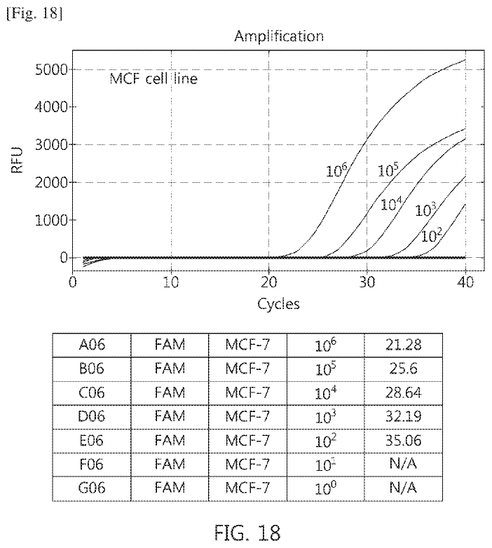

The sensitivity using RT-qPCR of HER2 with a base sequence (site 605) at another site of the front part disclosed in FIGS. 1 to 7 was verified by the sensitivity capable of detecting 10¹ of SK-BR-3 cells and 10² of MCF7 cells (see FIGS. 17 and 18). When comparing Ct values at sites 605 to 612, the sensitivity at site 612 was relatively high as 18.64 and 17.06, and 21.28 and 20.47 in 10⁶ of SKBR3 and MCF7 cell lines, respectively.

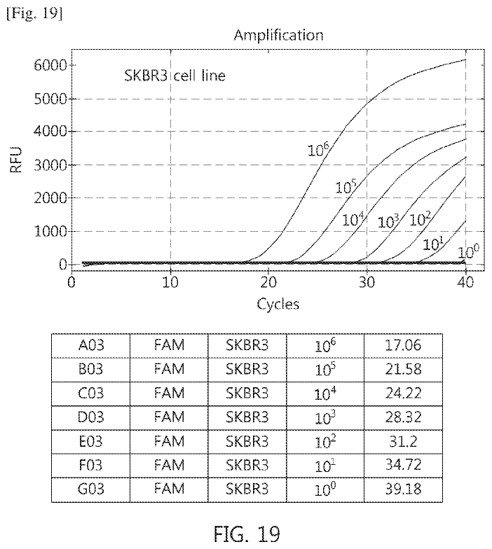

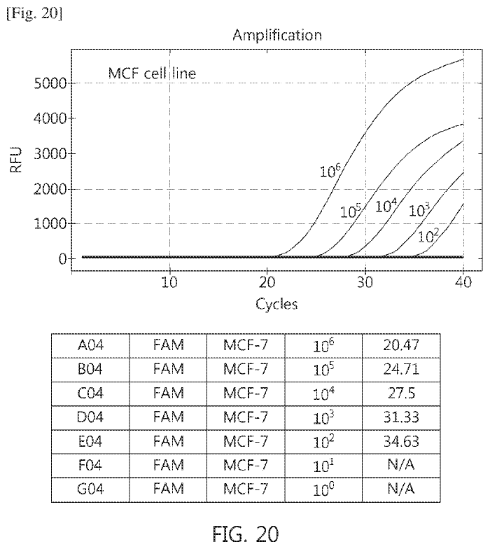

Further, the sensitivity using RT-qPCR of HER2 with a base sequence (site 612) of the front part illustrated in FIGS. 19 and 20 was verified by the sensitivity capable of detecting 10¹ of SK-BR-3 cells and 10² of MCF7 cells.

ii) Comparison of HER2 expression levels for each cell line using base sequence (site 2.7 kb) of middle part

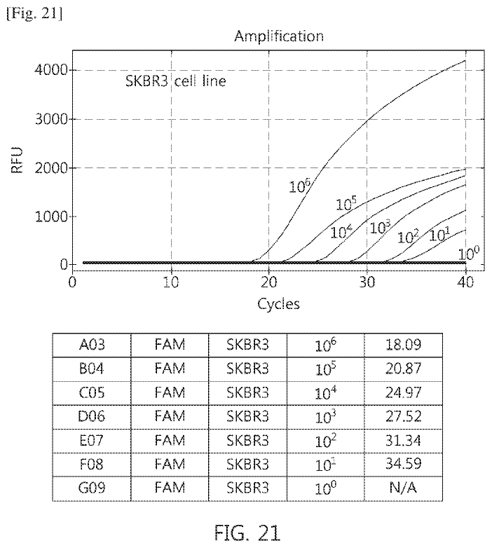

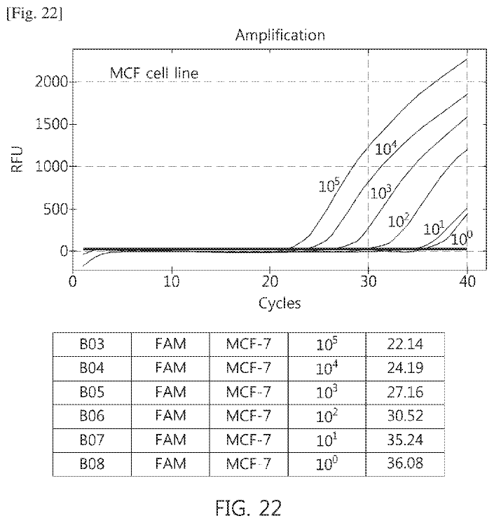

\textcircled{1} The sensitivity using RT-qPCR of HER2 with a primer and a probe p1 of a base sequence of a part 2725 positioned at the fourth box in FIGS. 8 to 16 was verified by the sensitivity capable of detecting 10¹ of SK-BR-3 cells and 10¹ of MCF7 cells (see FIGS. 21 and 22).

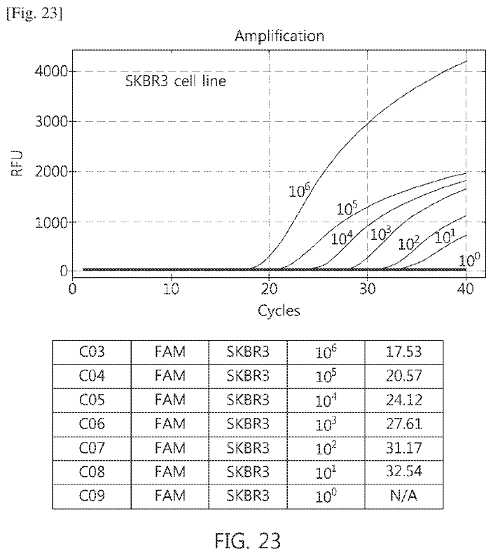

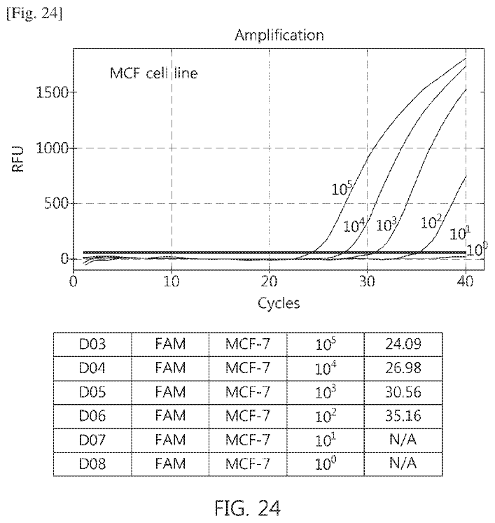

\textcircled{2} The sensitivity using RT-qPCR of HER2 with a primer and a probe p2 of a base sequence of a part 2725 was verified by the sensitivity capable of detecting 10¹ of SK-BR-3 cells and 10² of MCF7 cells (see FIGS. 23 and 24).

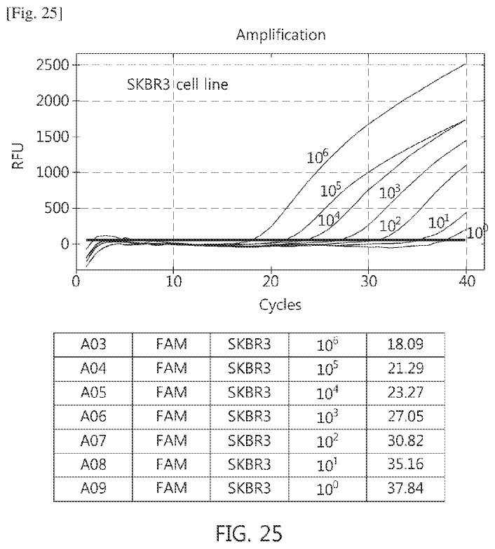

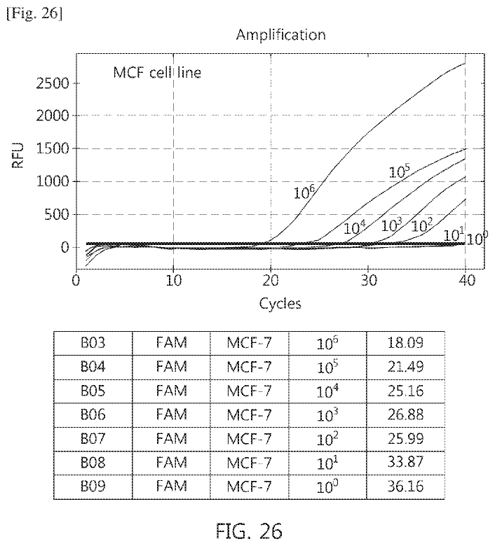

\textcircled{3} The sensitivity using RT-qPCR of HER2 with a primer and a probe p1-2mix of a base sequence of a part 2725 was verified by the sensitivity capable of detecting 10¹ of SK-BR-3 cells and 10¹ of MCF7 cells (see FIGS. 25 and 26).

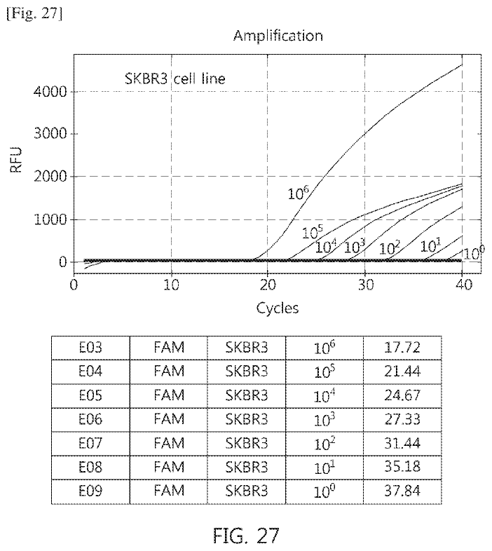

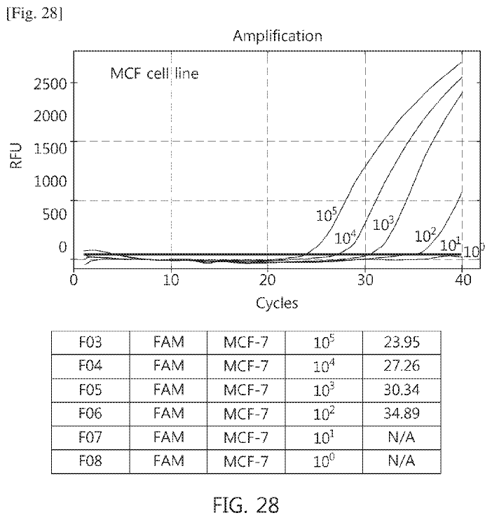

\textcircled{4} The sensitivity using RT-qPCR of HER2 with a primer and a probe p1 of a base sequence of a part 2740 positioned at the fourth box in FIG. 1B was verified by the sensitivity capable of detecting 10¹ of SK-BR-3 cells and 10² of MCF7 cells (see FIGS. 27 and 28).

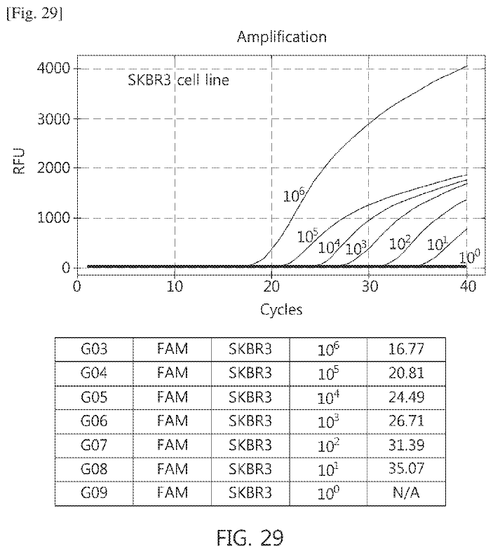

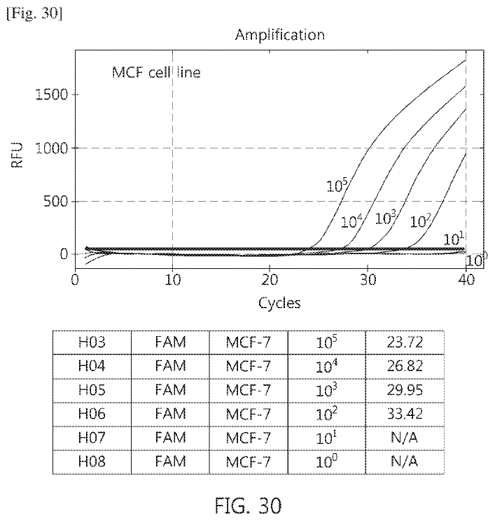

The sensitivity using RT-qPCR of HER2 with a primer and a probe p2 of a base sequence of a part 2740 was verified by the sensitivity capable of detecting 10¹ of SK-BR-3 cells and 10² of MCF7 cells (see FIGS. 29 and 30).

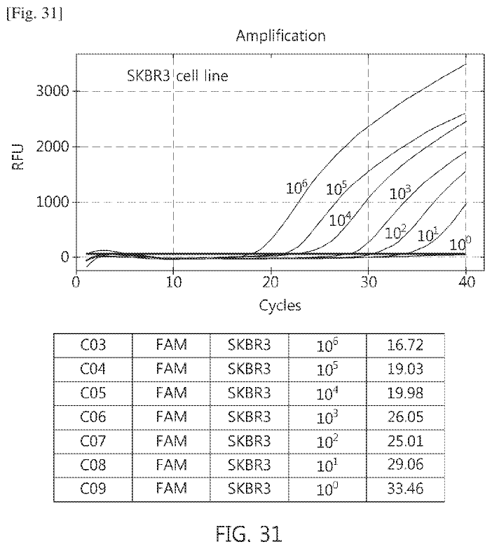

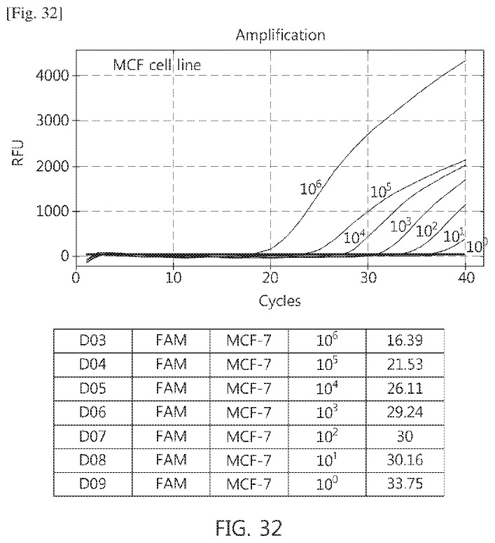

\textcircled{5} The sensitivity using RT-qPCR of HER2 with a primer and a probe P1-2mix of a base sequence of a part 2740 was verified by the sensitivity capable of detecting one of SK-BR-3 cells and 10¹ of MCF7 cells (see FIGS. 31 and 32).

When comparing Ct values at sites 2725 to 2740, by changing only the location of the probe while using the same primer at the site 2725, the sensitivity was 18.09/17.53/18.09 in 10⁶ of the SKBR3 and MCF7 cell lines of P1/P2/P1+2mix, respectively, and by changing only the location of the probe while using the same primer at the site 2740, the sensitivity was 17.72/16.77/16.72 in 10⁶ of the SKBR3 and MCF7 cell lines of P1/P2/P1+2mix, respectively. As a result, the sensitivity at the site 2740 is relatively higher than the sensitivity at the site 2725 and the sensitivity in P1+2mix is relatively higher than the sensitivity in P1 and P2.

As such, it was verified that the base sequences according to the site had different results and the sensitivity varied through the single RT-qPCR.

(2) Comparison of HER2 Expression Levels for Each Cell Line Using Multiplex RT-qPCR

In order to determine a change in sensitivity when mixing two base sequences compared with the single RT-qPCR prepared above, the expression sensitivity of HER2 was verified by using SK-BR-3 and MCF-7 which were HER2 positive breast cancer cell lines by mixing base sequences at the front part (sites 605 to 612) and the middle part (sites 2725 to 2740).

In the Single RT-qPCR, the sensitivity was high compared with the result when the PI-P2mix was put at sites 2725 to 2740, respectively, and performed by the same condition of P1-P2mix.

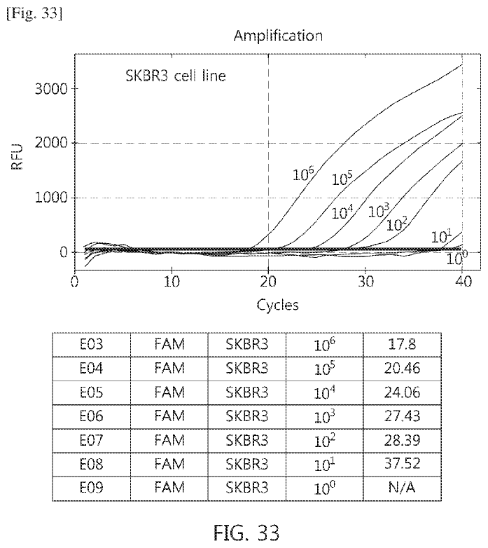

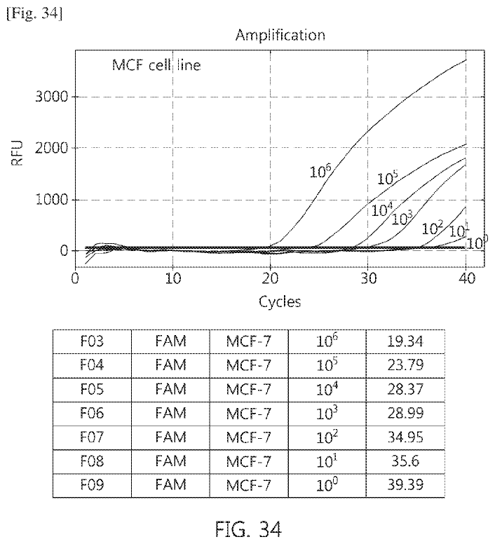

\textcircled{1} The sensitivity using RT-qPCR of HER2 with base sequences at the front part (site 605) and the middle part (site 2725) illustrated in FIGS. 33 and 34 was verified by the sensitivity capable of detecting 10¹ of SK-BR-3 cells and 10¹ of MCF7 cells.

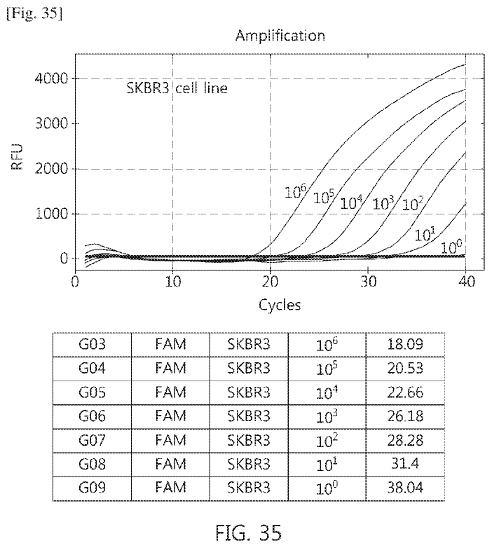

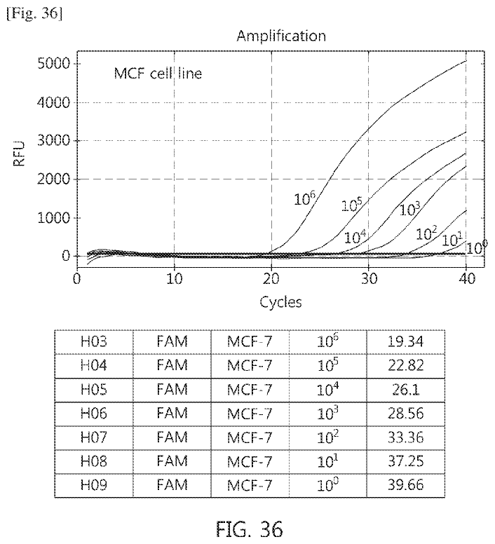

\textcircled{2} The sensitivity using RT-qPCR of HER2 with base sequences at the front part (site 612) and the middle part (site 2725) was verified by the sensitivity capable of detecting 10¹ of SK-BR-3 cells and 10¹ of MCF7 cells (see FIGS. 35 and 36).

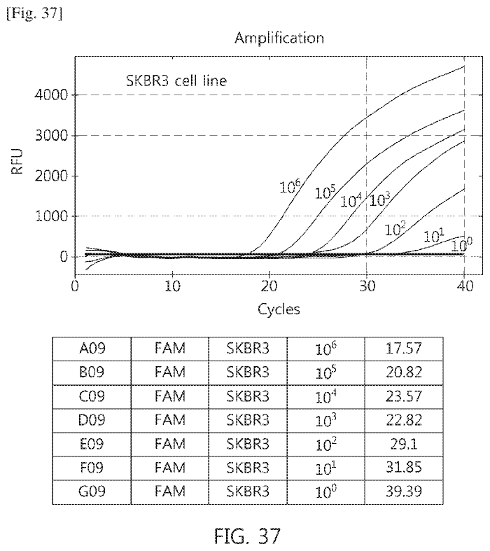

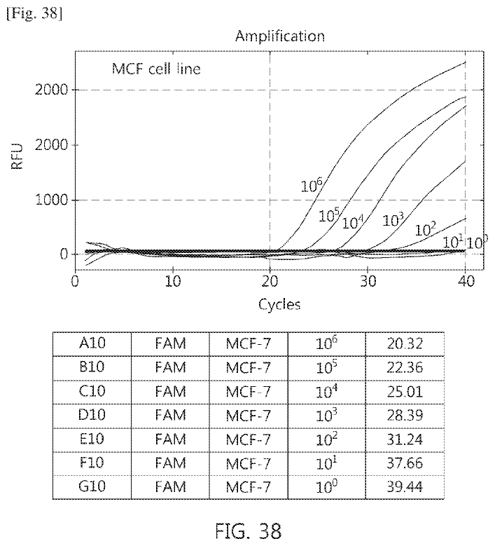

\textcircled{3} The sensitivity using RT-qPCR of HER2 with base sequences at the front part (site 605) and the middle part (site 740) was verified by the sensitivity capable of detecting 10¹ of SK-BR-3 cells and 10² of MCF7 cells (see FIGS. 37 and 38).

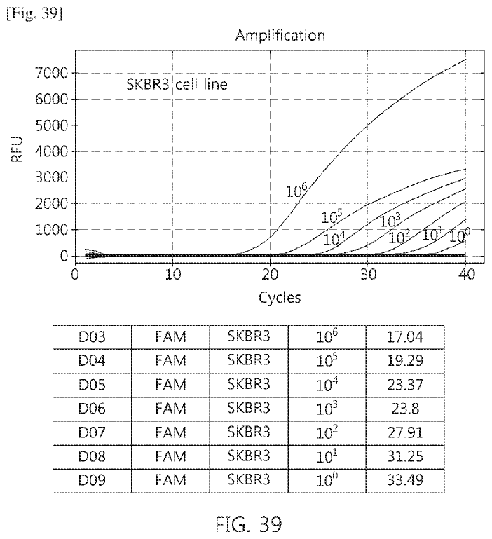

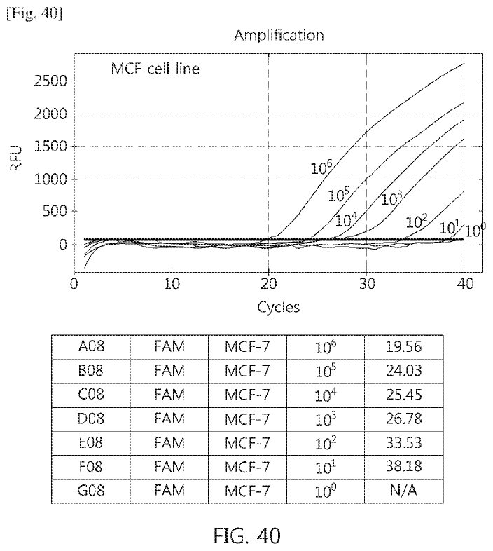

\textcircled{4} The sensitivity using RT-qPCR of HER2 with base sequences at the front part (site 612) and the middle part (site 2740) was verified by the sensitivity capable of detecting one of SK-BR-3 cells and 10² of MCF7 cells (see FIGS. 39 and 40).

In the case of the multiplex RT-qPCR, the sensitivity was increased compared with the single RT-qPCR performed above, but even in this case, the Ct values of the SKBR-MCF7 cell line obtained by mixing base sequences at site 612 of the front part were 17.04 and 19.56, whereas the Ct values of the SKBR-MCF7 cell line obtained by base sequences at site 605 were 17.57 and 20.32, and thus it was verified that the sensitivity was decreased and the result values varied according to which site base sequences were mixed with each other.

(3) Comparison of HER2 Expression Levels for Each Cell Line Using One-Tube Nested RT-qPCR

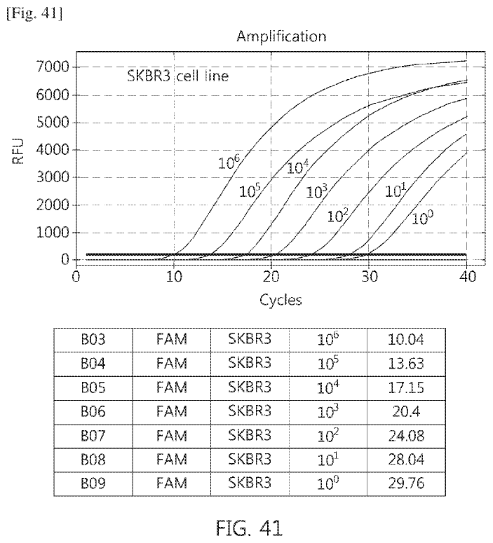

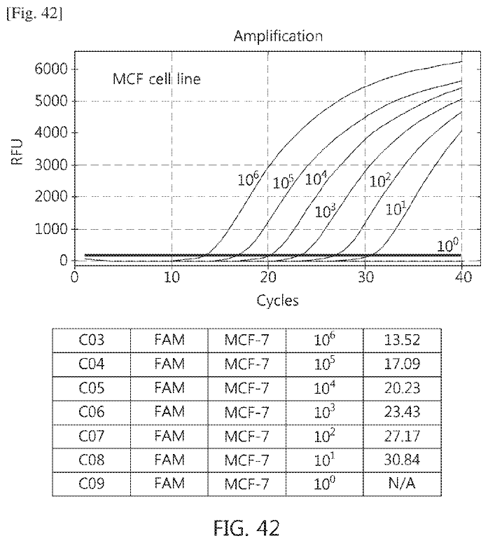

First, a change in sensitivity was determined by using the one-tube nested RT-qPCR method in order to increase the sensitivity compared with the results of the single RT-qPCR and the multiplex RT-qPCR prepared above. First, the expression sensitivity of HER2 was verified by using SK-BR-3 and MCF-7 which were HER2 positive breast cancer cell lines using base sequences at the front part (sites 605 to 612). As a result, the sensitivity capable of detecting one of the SK-BR-3 cells and 10¹ of the MCF7 cells was verified (see FIGS. 41 and 42).

In the case of the SKBR3 cell line (10⁶), the result (the Ct values were 8.64 and 17.57) of the single-multiplex RT-qPCR was verified, whereas in the case of the one-tube nested RT-qPCR, the high sensitivity was verified as 10.04.

Next, the expression sensitivity of HER2 was verified by using SK-BR-3 which was a HER2 positive breast cancer cell line of the one-tube nested RT-qPCR by using the base sequences at the middle part (2725 to 2740). A sensitivity test was performed by P1/P2/P1+2mix according to a site of the probe, respectively.

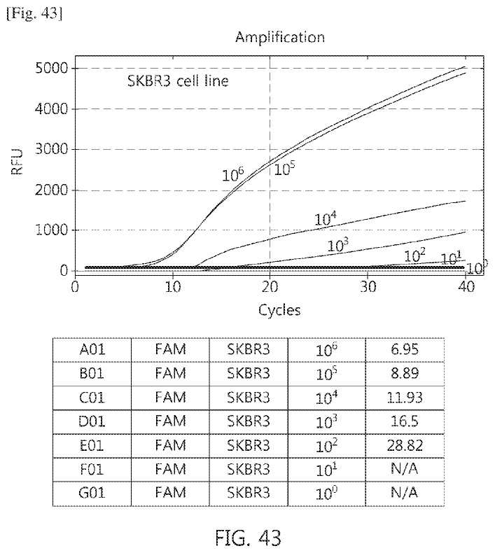

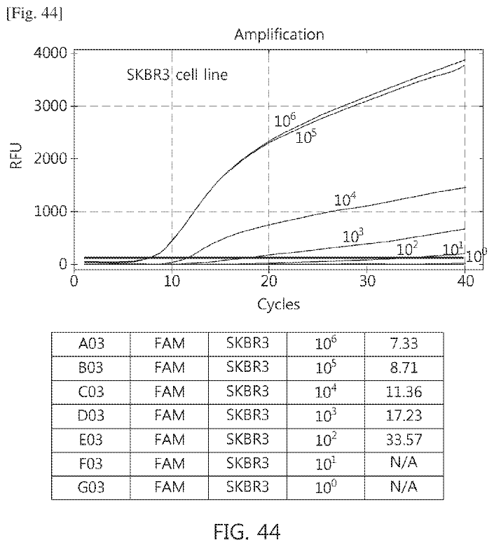

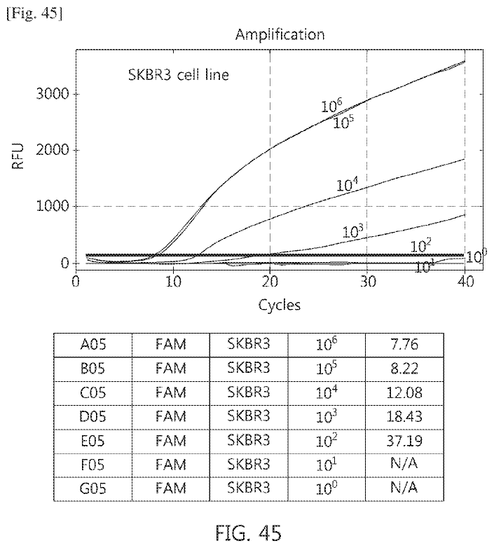

\textcircled{1} First, when showing the result when the probe P1 was used by using the SK-BR-3 cells, the Ct value of 10⁶ was 6.95 and thus the high sensitivity was shown, but detected only in 10² cells (see FIG. 43). Even in the case of using the probe P2, similarly, the Ct value of 10⁶ was 7.33 and thus the high sensitivity was shown, but detected only in 10² cells (see FIG. 44). As a result of verifying the sensitivity by mixing P1 and P2, the Ct value of 10⁶ was 7.76 and thus the high sensitivity was shown, but detected only in 10² cells (see FIG. 45).

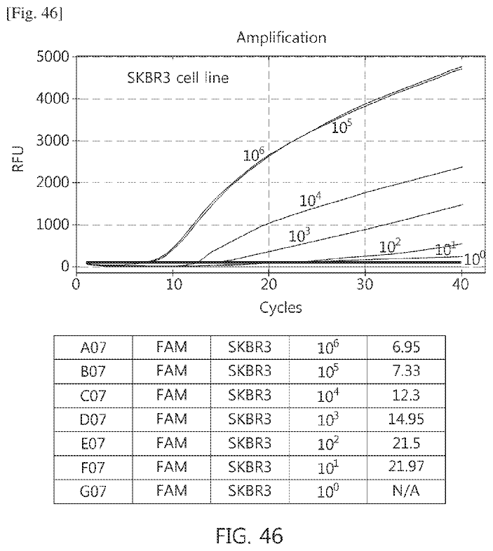

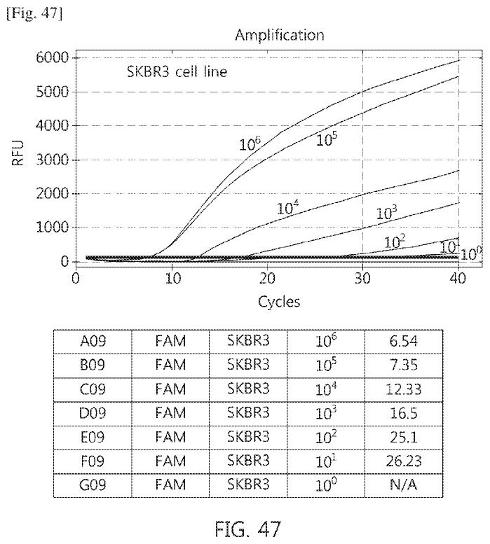

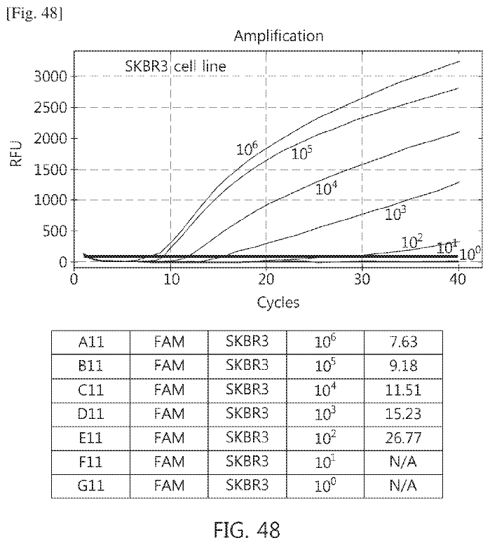

\textcircled{2} HER2 expression levels of one-tube nested RT-qPCR were compared by mixing base sequences at the front part (605 to 612) and the middle part (2725 to 2740). First, when showing the result when the probe P1 was used, the Ct value of 10⁶ was 6.95 and thus the high sensitivity was shown, but detected only in 10¹ cells (see FIG. 46). Even in the case of using the probe P2, similarly, the Ct value of 10⁶ was 6.54 and thus the high sensitivity was shown, but detected only in 10¹ cells (see FIG. 47). As a result of verifying the sensitivity by mixing P1 and P2, the Ct value of 10⁶ was 7.63 and thus the high sensitivity was shown, but detected only in 10² cells (see FIG. 48).

\textcircled{3} The base sequences at the front part (605 to 612) were used as it is and the HER2 expression levels of one-tube nested RT-qPCR were compared by mixing two probes (P1-2) in a pair of primers at the middle part.

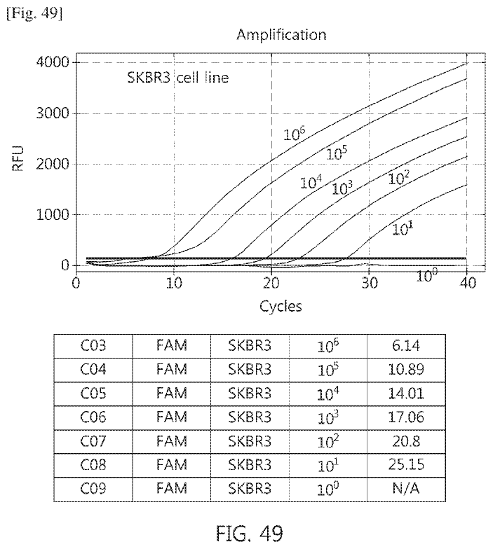

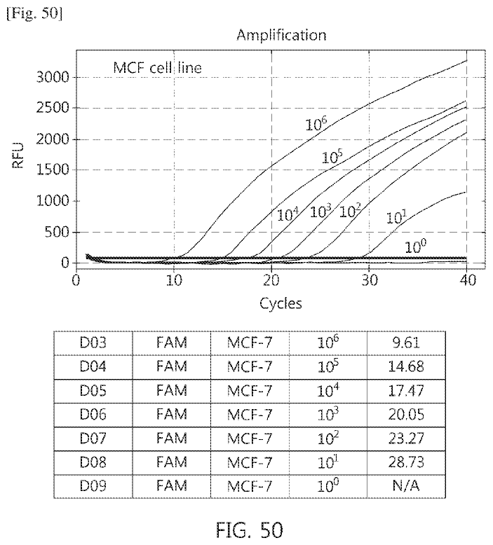

First, as a result of verifying the expression sensitivity of HER2 by using the SK-BR-3 and the MCF-7 by mixing the middle part (2725), the Ct values of 10⁶ were 6.14 and 9.61, and thus the high sensitivity was shown and the entire sensitivity was detected up to one and 10¹ of the cells (see FIGS. 49 and 50).

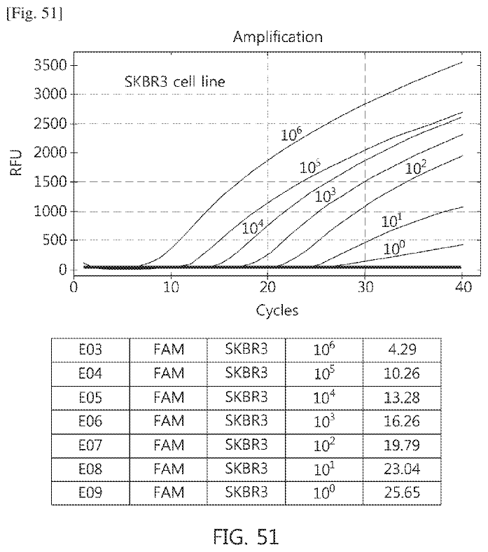

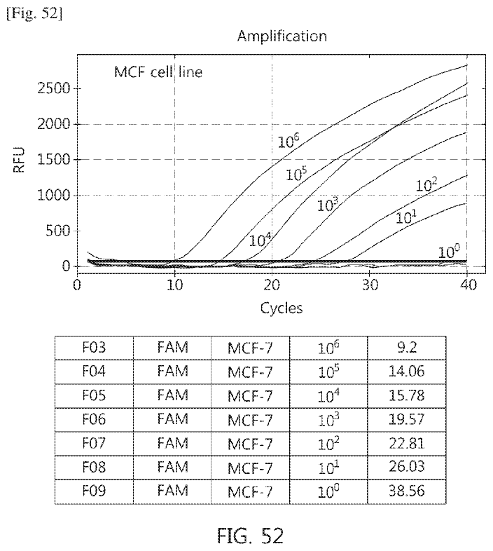

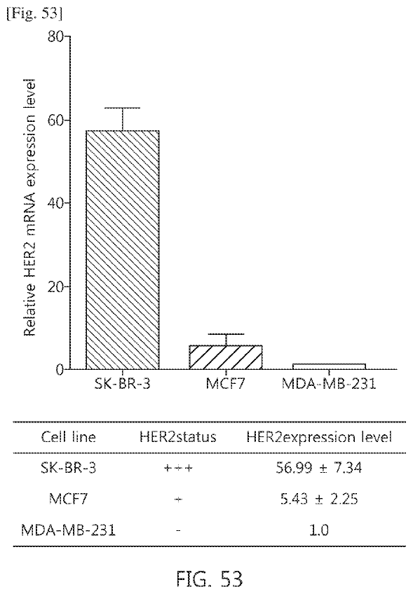

In the result of mixing the middle part (2740), the Ct values of 10⁶ were 4.29 and 9.2, and thus the high sensitivity was shown and the entire sensitivity was detected up to 10¹ and 10² of the cells (see FIGS. 51 and 52).

As a result of summarizing the test performed above according to three methods, in the Single RT-qPCR, the HER2 expression levels shown according to sites of the primer and the probe varied, and at the front part, the sensitivity of site 612 was higher than that of site 605, at the middle part, the sensitivity of 2740 was higher than that of 2725, and the sensitivity when combining the probes P1 and P2 was higher than that of either P1 or P2 (the Ct values in 10⁶-10⁵-10⁴-10³-10²-10¹-10⁰ were 16.72-18.96-20.23-23.27-25.79-26,7-34.27, respectively). The result showed the same trend even though the cell line varied (see Tables 1 and 2).

Further, in the case of the multiplex RT-qPCR, the sensitivity when mixing the base sequences at sites 612 to 2740 was higher than that of other three cases of sites 605 to 2725, 612 to 2725, and 605 to 2740 (the Ct values in 10⁶-10⁵-10⁴-10³-10²-10¹-10⁰ were 17.04-19.29-23.37-23.8-27.91-31.25-33.49, respectively).

Even in the method using the One-tube nested RT-qPCR condition, in the single or multiplex RT-OCR above, an obtained value varied according to which site the primer or the probe was mixed, and even in the one-tube nested RT-qPCR, the value was shown. In the one-tube nested RT-qPCR method itself, in the result of the Single RT-qPCR of the multiplex RT-qPCR, the sensitivity was better, but when mixing the base sequences at sites 2725 and 2740 of the middle part, the result value was still not good. Accordingly, the optimal condition of the one-tube nested RT-qPCR method of the present disclosure was shown as the front part (605-612)-2740-P1-2mix, and when comparing the respective Ct values, it was verified that in 10⁶-10⁵-10⁴-10³-10²-10¹-10⁰, the sensitivity was increased by 10⁴ to 10⁶ or more as 4.29-10.26-13.28-16.26-19.79-23.04-25.65 by Single RT-qPCR<multiplex RT-qPCR<One-tube nested RT-qPCR (see Table 3).

TABLE 1

| | | | | | | | | | Multiplex RT-qPCR (Ct) | | | |
|---|---|---|---|---|---|---|---|---|---|---|---|---|
| Single RT-qPCR (Ct) | | | | | | | | | 605- | 612- | 605- | 612- |
| | | | 2725- | 2725- | 2725- | 2740- | 2740- | 2740- | 2725- | 2725- | 2740- | 2740- |
| SKBR3 | 605 | 612 | P1 | P2 | P1-2 | P1 | P2 | P1-2 | P1-2 | P1-2 | P1-2 | P1-2 |
| 10⁶ | 18.64 | 17.06 | 18.09 | 17.53 | 18.09 | 17.72 | 16.77 | 16.72 | 17.8 | 18.09 | 17.57 | 17.04 |
| 10⁵ | 22.56 | 21.58 | 20.87 | 20.57 | 21.29 | 21.44 | 20.81 | 18.96 | 20.46 | 20.53 | 20.82 | 19.29 |
| 10⁴ | 25.55 | 24.22 | 24.97 | 24.12 | 23.27 | 24.67 | 24.49 | 20.23 | 24.06 | 22.66 | 23.57 | 23.37 |
| 10³ | 29.32 | 28.32 | 27.52 | 27.61 | 27.05 | 27.33 | 26.71 | 23.27 | 27.43 | 26.18 | 22.82 | 23.8 |
| 10² | 32 | 31.2 | 31.34 | 31.17 | 30.82 | 31.44 | 31.39 | 25.79 | 28.39 | 28.28 | 29.1 | 27.91 |
| 10¹ | 35.16 | 34.72 | 34.59 | 32.54 | 35.16 | 35.18 | 35.07 | 26.7 | 37.52 | 31.4 | 31.85 | 31.25 |
| 10⁰ | N/A | 39.18 | N/A | N/A | 37.84 | 37.84 | N/A | 34.27 | N/A | 38.04 | 39.39 | 33.49 |

TABLE 2

| | | | | | | | | | Multiplex RT-qPCR (Ct) | | | |
|---|---|---|---|---|---|---|---|---|---|---|---|---|
| Single RT-qPCR (Ct) | | | | | | | | | 605- | 612- | 605- | 612- |
| | | | 2725- | 2725- | 2725- | 2740- | 2740- | 2740- | 2725- | 2725- | 2740- | 2740- |
| MCF7 | 605 | 612 | P1 | P2 | P1-2 | P1 | P2 | P1-2 | P1-2 | P1-2 | P1-2 | P1-2 |
| 10⁶ | 21.28 | 20.47 | | | 18.09 | | | 16.39 | 19.34 | 19.34 | 20.32 | 19.56 |
| 10⁵ | 25.6 | 24.71 | 22.14 | 24.09 | 21.49 | 23.95 | 23.72 | 21.53 | 23.79 | 22.82 | 22.36 | 24.03 |
| 10⁴ | 28.64 | 27.53 | 24.19 | 26.98 | 25.16 | 27.26 | 26.82 | 26.11 | 28.37 | 26.1 | 25.01 | 25.45 |
| 10³ | 32.19 | 31.33 | 27.16 | 30.56 | 26.88 | 30.34 | 29.95 | 29.24 | 28.99 | 28.56 | 28.39 | 26.78 |
| 10² | 35.06 | 34.63 | 30.52 | 35.16 | 25.99 | 34.89 | 33.42 | 30 | 34.95 | 33.36 | 31.24 | 33.53 |
| 10¹ | N/A | N/A | 35.24 | N/A | 33.87 | N/A | N/A | 33.16 | 35.6 | 37.25 | 37.66 | 38.18 |
| 10⁰ | N/A | N/A | 36.08 | N/A | 36.16 | N/A | NIA | 33.75 | 39.39 | 39.66 | 39.44 | N/A |

Tables 1 and 2 list comparisons of HER2 expression levels according to single-multiplex RT-qPCR.

TABLE 3
one-tube nested RT-qPCR (Ct)

| | | | | 2725- | 605(612)- | 605(612)- | 605(612)- | 605(612)- | 605(612)- |
|---|---|---|---|---|---|---|---|---|---|
| | | 2725- | 2725- | 2740- | 2725(2740)- | 2725(2740)- | 2725(2740)- | 2725- | 2740- |
| SKBR3 | 605-612 | 2740-P1 | 2740-P2 | P1-2mix | P1 | P2 | P1-2mix | P1-2mix | P1-2mix |
| 10⁶ | 10.04 | 6.95 | 7.33 | 7.76 | 6.95 | 6.54 | 7.63 | 6.14 | 4.29 |
| 10⁵ | 13.63 | 8.89 | 8.71 | 8.22 | 7.33 | 7.35 | 9.18 | 10.89 | 10.26 |
| 10⁴ | 17.15 | 11.93 | 11.36 | 12.08 | 12.3 | 12.33 | 11.51 | 14.01 | 13.28 |
| 10³ | 20.4 | 16.5 | 17.23 | 18.43 | 14.95 | 16.5 | 15.23 | 17.06 | 16.26 |
| 10² | 24.08 | 28.82 | 33.57 | 37.19 | 21.5 | 25.1 | 26.77 | 20.8 | 19.79 |
| 10¹ | 28.04 | N/A | N/A | N/A | 21.97 | 26.23 | N/A | 25.15 | 23.04 |
| 10⁰ | 29.76 | N/A | N/A | N/A | N/A | N/A | N/A | N/A | 25.65 |

Table 3 lists a comparison of HER2 expression levels according to one-tube nested RT-qPCR.

Comparison of Expression Levels of HER2 mRNA Using Breast Cancer Cell Lines

Expression levels of HER2 in respective cell lines were compared by using SK-BR3, MCF7, and MDA-MB-231 cell lines as breast cancer cell lines. When the HER2 expression of the MDA-MB-231 cell line as a HER2 negative cell line was set as 1, it was verified that the HER2 expression level of the MCF7 was shown as approximately 5.4 and the HER2 expression level of the SK-BR-3 was shown as approximately 56.9.

Setting of Clinical Cut-Off

After HER2 RT-qPCR was performed by using FFPE specimens of 199 breast cancer patients provided from Severance Hospital in Shinchon, the result was compared with the IHC Score and the FISH result of the breast cancer patients. Scores were designated to 0 in the case of IHC 0, 25 in the case of IHC 1+, 50 in the case of IHC 2+ and FISH negative, 75 in the case of IHC 2+ and FISH positive, and 100 in the case of IHC 3+, respectively, and as a result, the results of the HER2 RT-qPCR and the one-tube nested RT-qPCR were compared with each other.

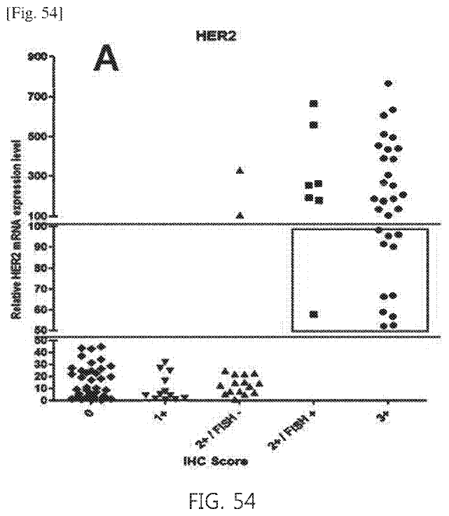

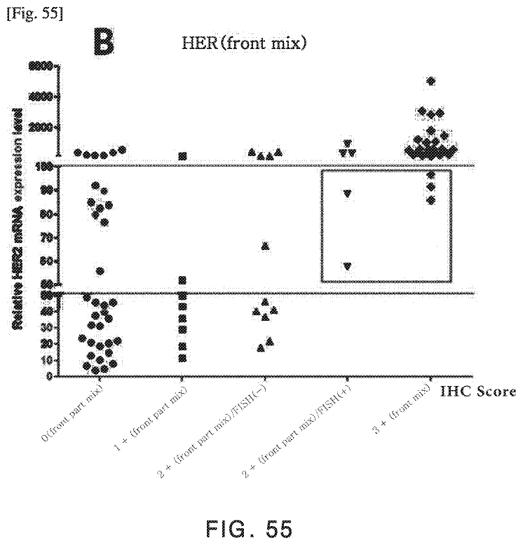

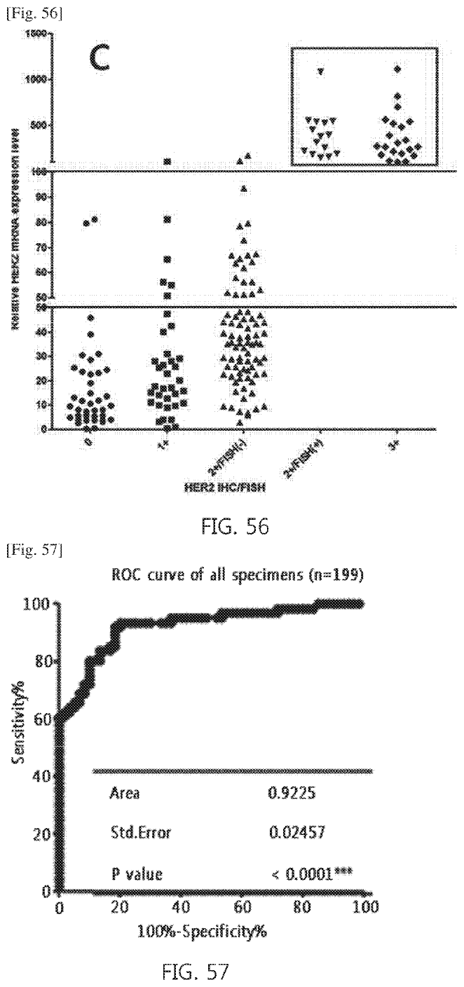

FIG. 54 illustrates a comparison of expression levels using the single RT-qPCR, FIG. 55 illustrates a comparison of expression levels using the multiplex RT-qPCR, and FIG. 56 illustrates a comparison of expression levels using one-tube nested RT-qPCR. In blue boxes in the drawings, the expression levels were distributed on a boundary of negative and positive in the case of patient specimens verified as the IHC 2+/FISH positive and the IHC 3+/HER2 positive or some of specimens in the case of the single RT-qPCR and the multiplex RT-qPCR, but in the case of the one-tube nested RT-qPCR condition, the result of the positive expression was shown.

Even in the clinical evaluation, it was verified that in the case of the one-tube nested RT-qPCR condition, the sensitivity was increased as compared with other two methods.

Clinical Result Analysis Using ROC Curve

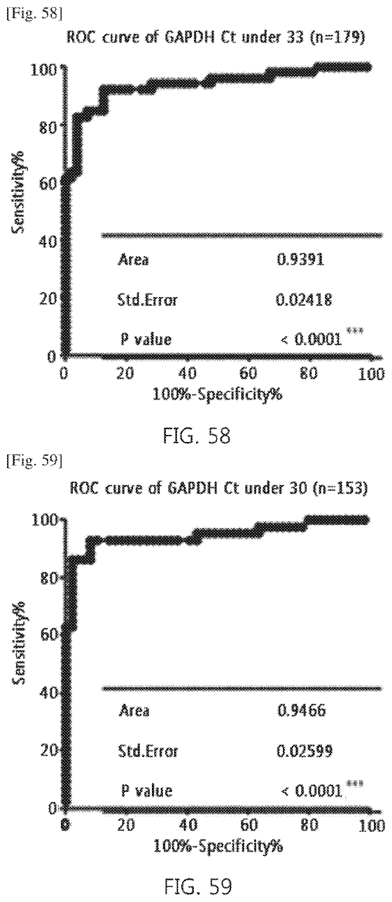

In the test using the FFPE specimens, the RNA quality of the specimens was very important. The specimens having the high RNA quality may show the accurate result, while if the RNA quality is low, the result of false-positive or false-negative may be shown. Accordingly, in the present disclosure, the degree of the RNA quality was indicated based on the expression level of GAPDH. As illustrated in FIGS. 57 to 59, it was verified that when the expression degree of GAPDH was classified based on the Ct value of RT-qPCR, as the Ct value of GAPDH had a low value, a more accurate result was shown.

In this case, a receiver operating characteristic (ROC) curve is a graph shoving performance of a determined result (binary classifier) of any test, and

has a true positive rate (TPR) or sensitivity as a y axis and a false positive rate (FPR) or 1-specificity as an x axis.

That is, the result is calculated as TRP=y axis=sensitivity=(TP/(TP+FN) and FPR=x axis=1-specificity=1−[TN/(TN+FP)].

TABLE 4

| posi- | | | Modifi- |
|---|---|---|---|
| tion | name | sequence (5′-3′) | cation |
| 605 | HER605-F | AACCTGGAACTCACCTACCTGCCCAC | |
| | | (SEQ ID NO. 1) | |
| | HER689-R | CGATGAGCACGTAGCCCTGCAC (SEQ | |
| | | ID NO. 2) | |
| 612 | HERE12-F | AACTCACCTACCTGCCCACCAAT | |
| | | (SEQ ID NO. 3) | |
| | HER680-R | CACGTAGCCCTGCACCTCCT(SEQ ID | |
| | | NO. 4) | |
| | HER637-P | CAGCCTGTCCTTCCTGCAGGATATC | FAM- |
| | | (SEQ ID NO. 5) | BHQ1 |
| 2725 | HER2725- | AGAAATCTTAGACGAAGCATACGTG | |
| | F | AT(SEQ ID NO. 6) | |
| | HER2865- | TCCCGGACATGGTCTAAGAGGCA | |
| | R | (SEQ ID NO. 7) | |
| 2740 | HER2740- | AAGCATACGTGATGGCTGGTG T (SEQ | |
| | F1 | ID NO. 8) | |
| | HER2853- | TCTAAGAGGCAGCCATAGGGCATA | |
| | R1 | (SEQ ID NO. 9) | |
| | HER2-P1 | ATATGTCTCCCGCCTTCTGGGCATCT | FAM- |
| | | (SEQ ID NO. 10) | BHQ1 |
| | HER2-P2 | CATCCACGGTGCAGCTGGTGACACA | FAM- |
| | | (SEQ ID NO. 11) | BHQ1 |
| | GAPDH-F | CCATCTTCCAGGAGCGAGATCC(SEQ | |
| | | ID NO. 12) | |
| | GAPDH-R | ATGGTGGTGAAGACGCCAGTG(SEQ | |
| | | ID NO. 13) | |
| | GAPDH-P | TCCACGACGTACTCAGCGCCAGCA | Cy5- |
| | | (SEQ ID NO. 14) | BHQ2 |

Table 4 lists a base sequence list used in the present disclosure.

Hereinafter, various exemplary embodiments of the present disclosure will be described in detail with reference to the accompanying drawings. However, the present disclosure is not limited to the exemplary embodiments disclosed below, but can be implemented in various forms. The following exemplary embodiments are described in order to enable those of ordinary skill in the art to embody and practice the invention.

### Example 1: Materials

Formalin fixed paraffin embedded (FFPE) tissues of 199 patients were used at Severance Hospital in Shinchon from 2010 to 2011. Expression of histological HER2 of the patients was verified by performing immunohistochemistry (IHC) and fluorescence in situ hybridization (FISH). Further, in order to verify the expression of HER2, the expression of HER2 was verified by using SK-BR3, MCF7, and MDA-MB 231 as breast cancer cell lines.

### Example 2: Immunohistochemistry (IHC)

A paraffin block was thin-sectioned with a thickness of 4 μm to be attached to a slide and sufficiently dried, and then immunohistochemistry (IHC) was performed by using a BenchMark ST (Ventana medical system, USA) automatic immunostaining device. A primary antibody was used by diluting polyclonal rabbit anti-human c-erbB-2 oncoprotein (A0485, DakoCytomation, Glostrup, Denmark) with 1:1.000. After the slide was stained by the method, it was determined that the stained slide was divided into four grades, that is, 0, 1+, 2+, and 3+ according to the degree that a HER2 protein was stained in a cell membrane of a cancer cell. Among the four grades, 0 and 1+ were diagnosed as HER2 negative, 3+ was diagnosed as positive, and 2+ was diagnosed as positive or by performing FISH according to clinical information of the patient.

### Example 3: Fluorescence in Situ Hybridization (FISH)

In a HER2 IHC method, by targeting patients showing 2+, after a tissue block fixed with paraffin was thin-sectioned with a thickness of 4 μm by using a microtome to be attached to a slide, a test was performed according to a manufacturer's instructions by using a HER2 DNA probe kit (Vysis Downers Grove, Ill. USA) commercialized through deparaffinization and hydration processes. The HER2 expression was determined as positive according to a gene expression degree when an amplification index was 2.2 or more.

### Example 4: Total RNA Isolation from Isolated Tissue

RNA was extracted by using a MagNApure LC RNA isolation Kit III (Roche) as automatic nucleic acid extraction equipment after performing the deparaffinization process by using two pieces obtained by thin-sectioning a FFPE tissue with a thickness of 10 μm.

In the case of a cell line, after the number of cells in each cell line was adjusted to 1×10⁶, Total RNA was isolated by using Trizol according to a protocol of a manufacturer. The isolated Total RNA was quantified by using a NanoQuant system (TECAN).

### Example 5: cDNA Preparation from Isolated Total RNA and Performance of Real-Time PCR

i. cDNA Synthesis

0.5 to 3 μg of the isolated total RNA, 0.25 μg of random primer (Invitrogen), 250 μM of dNTP (Intron), Tris-HCl (pH 8.3) 50 mM, KCl 75 mM, MgCl₂ 3 mM, DTT 8 mM, and MMLV reverse transcription polymerase 200 units (Invitrogen) were added and mixed with DEPC-treated DW to have 30 μl of a final volume, and then reacted with a synthesis reaction solution for 10 mins at 25° C., for 50 mins at 37° C., and for 15 mins at 70° C. in a thermocycler (ABI) to synthesize cDNA.

ii. Performance of RT-qPCR

A composition of a reactant of Real-time PCR was prepared by adding 25 mM TAPS (pH 9.3 at 25° C.), 50 mM KCl, 2 mM MgCl₂, 1mM 2-mercaptoethanol, 200 μM each dNTP, and 1 unit Taq polymerase (TAKARA), adding 10 mole of a forward primer and a reverse primer, respectively, adding 10 pmole of probe, and adding 2 μl of the synthesized cDNA to have a final volume of 20 μl. Respective primers and a base sequences of probes are disclosed in Table 4.

The PCR reaction used CFX 96 (Bio-rad, USA) and was performed by a method of two denaturing temperatures.

The Single RT-PCR was performed once for 3 mins at 94° C. and a cycle which was 30 secs at a denaturing temperature of 95° C. and 40 secs at an annealing temperature of 55° C. was repetitively performed 40 times.

The one-tube nested RT-PCR was performed once for 3 mins at 94° C. 10 cycles was first performed for 30 secs at a denaturing temperature of 95° C. and an annealing temperature of 60° C. and then a cycle which was 30 secs at 95° C. and 40 secs at 55° C. was repetitively preformed 40 times.

Further, a process of measuring fluorescence was added after each annealing process to measure an increased fluorescence value for each cycle.

### Example 6: Analysis of Result

The result of each test was analyzed by using Bio-Rad CFX manager v1.6 (Bio-Rad). SK-BR3 and MCF7 as breast cancer cells were diluted from 10⁶ to 1 cell in stages to draw a relative quantitative curve, and then expression levels were compared and quantified by using a Ct value to examine an expression rate. In this case, expression levels of each HER2 were compared based on an expression level of GAPDH, and an expression level of HER2 in each specimen and each cell line was indicated after determining a HER2 expression level of MDA-MB-231 as a HER2 negative breast cancer cell line as a reference of 1.

### Example 7: Verification of Amplification Through Software Analysis and Quantification of Amplified Product

An expression level of a specific gene of qRT-PCR was measured based on the following Relational Formula by using a comparative Ct method which was one of the quantifying methods and the Formula was embedded in Bio-Rad CFX Manager Software and automatically calculated.

ΔΔCt=ΔCt (sample)−ΔCt (reference gene) [Relational Formula 1]

Herein, the Ct value represented a value of cycle in which amplification started to be distinctly increased during the PCR process.

ΔΔCt means a mRNA expression ratio of a vertical axis in FIG. 3 below.

Relational Formula of expression level analysis of HER2 in positive control group

ΔCt value of SKBR3=Ct value of HER2 in SKBR3−Ct value of reference gene (GAPDH) in SKBR3

ΔCt value of THP-1=Ct value of HER2 in THP-1−Ct value of reference gene (GAPDH) in THP-1

R (expression level)=ΔCt value of SKBR3−ΔCt value of THP-1 [Relational Formula 2]

Relational Formula of expression level analysis of HER2 for tissue sample of breast cancer patient

ΔCt value in breast cancer patient tissue=Ct value of HER2 in breast cancer patient tissue−Ct value of reference (GAPDH) gene in tissue

ΔCt value of THP-1=Ct value of HER2 in THP-1−Ct value of reference gene (GAPDH) in THP-1

R (expression level)=ΔCt value in breast cancer patient tissue−ΔCt value in THP-1 [Relational Formula 3]

The Ct value of the reference gene used in the test represented the Ct value for GAPDH and the reference gene may include another house keeping gene in addition to GAPDH used in this test.

SKBR3: it can be verified whether HER2 is actually overexpressed as a positive control.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for providing information for diagnosis of breast cancer, the method comprising:
a) isolating a total RNA from cells obtained from the blood of a cancer suspected patient;
b) synthesizing cDNA from the isolated total RNA;
c) performing a realtime-PCR of the synthesized cDNA by using one or more primer sets and probes selected from the group consisting of a primer set and a probe capable of amplifying a human epidermal growth factor receptor 2 HER2 and a primer set and a probe capable of amplifying glyceraldehyde-3-phosphate dehydrogenase (GAPDH); and
d) comparing the amplified level with an expressed level in a normal person;
wherein the primer set capable of amplifying the HER2 is a primer set selected from the group consisting of primer sets as set forth in SEQ ID NOS. 1 and 2, SEQ ID NOS. 3 and 4, SEQ ID NOS. 6 and 7, and SEQ ID NOS. 8 and 9 or a mixture of these primer sets and the probe is one or more of probes as set forth in SEQ ID NO. 5, SEQ ID NO. 10, and SEQ ID NO. 11.

2. The method for providing information for diagnosis of breast cancer of claim 1, wherein the comparing of the amplified level with the amplified level in the normal person is performed by a standard or cut-off value.

3. The method for providing information for diagnosis of breast cancer of claim 1, wherein the primer set capable of amplifying the GAPDH is set forth in SEQ ID NOS. 12 and 13 and the probe has a base sequence as set forth in SEQ ID NO. 14.

4. A composition for diagnosing breast cancer, the composition comprising:
a primer sets capable of amplifying a HER2 which is selected from the group consisting of primer sets as set forth in SEQ ID NOS. 1 and 2, SEQ ID NOS. 3 and 4, SEQ ID NOS. 6 and 7, and SEQ ID NOS. 8 and 9 or a mixture of these primer sets; and
a primer set and a probe capable of amplifying GAPDH, as active ingredients.

5. The composition for diagnosing breast cancer of claim 4, wherein the primer set capable of amplifying the GAPDH is set forth in SEQ ID NOS. 12 and 13 and the probe has a base sequence as set forth in SEQ ID NO.14.

6. (canceled)

7. (canceled)

8. The composition for diagnosing breast cancer of claim 4, wherein a 5′-terminal of the probe is marked with a fluorescent material.

9. The composition for diagnosing breast cancer of claim 5, wherein a 5′-terminal of the probe is marked with a fluorescent material.

10. A kit for diagnosing breast cancer, the kit comprising the composition of claim 4.

11. A kit for diagnosing breast cancer, the kit comprising the composition of claim 5.


SEQUENCE LISTING (14 sequences)

SEQ ID NO: 1 - DNA, 26 bp, Artificial Sequence
  primer
aacctggaactcacctacctgcccac

SEQ ID NO: 2 - DNA, 22 bp, Artificial Sequence
  primer
cgatgagcacgtagccctgcac

SEQ ID NO: 3 - DNA, 23 bp, Artificial Sequence
  primer
aactcacctacctgcccaccaat

SEQ ID NO: 4 - DNA, 20 bp, Artificial Sequence
  primer
cacgtagccctgcacctcct

SEQ ID NO: 5 - DNA, 25 bp, Artificial Sequence
  probe
cagcctgtccttcctgcaggatatc

SEQ ID NO: 6 - DNA, 27 bp, Artificial Sequence
  primer
agaaatcttagacgaagcatacgtgat

SEQ ID NO: 7 - DNA, 23 bp, Artificial Sequence
  primer
tcccggacatggtctaagaggca

SEQ ID NO: 8 - DNA, 22 bp, Artificial Sequence
  primer
aagcatacgtgatggctggtgt

SEQ ID NO: 9 - DNA, 24 bp, Artificial Sequence
  primer
tctaagaggcagccatagggcata

SEQ ID NO: 10 - DNA, 26 bp, Artificial Sequence
  probe
atatgtctcccgccttctgggcatct

SEQ ID NO: 11 - DNA, 25 bp, Artificial Sequence
  probe
catccacggtgcagctggtgacaca

SEQ ID NO: 12 - DNA, 22 bp, Artificial Sequence
  primer
ccatcttccaggagcgagatcc

SEQ ID NO: 13 - DNA, 21 bp, Artificial Sequence
  primer
atggtggtgaagacgccagtg

SEQ ID NO: 14 - DNA, 24 bp, Artificial Sequence
  probe
tccacgacgtactcagcgccagca